(19) United States
(12) Patent Application Publication
(10) Pub. No.: US 2026/0240948 A1
(43) Pub. Date: Aug. 20, 2026

(54) RECOMBINANT HUMAN ACIDIC FIBROBLAST GROWTH FACTOR GEL AND PREPARATION PROCESS THEREFOR

(71) Applicant: SHANGHAI TENRY PHARMACEUTICAL CO., LTD., Shanghai (CN)

(72) Inventors: Guoxing ZHU, Shanghai (CN); Dongjun FU, Shanghai (CN); Fang TANG, Shanghai (CN); Lufan GUAN, Shanghai (CN); Zhengwu LI, Shanghai (CN); Jiaxin HE, Shanghai (CN)

(21) Appl. No.: 19/156,028

(22) PCT Filed: Jan. 3, 2024
(86) PCT No.: PCT/CN2024/070258

§ 371 Date: Aug. 13, 2025

(30) Foreign Application Priority Data
Jun. 27, 2023 (CN) 202310760678.9

(51) Int. Cl. A61K 38/18; A61K 9/06; A61K 47/22; A61K 47/32; A61K 47/36

(52) U.S. Cl. CPC A61K 38/1825; A61K 9/06; A61K 47/22; A61K 47/32; A61K 47/36

(57) ABSTRACT

A recombinant human acidic fibroblast growth factor (aFGF) gel. The gel comprises a gel matrix, a thickener, and a protectant. The gel matrix is selected from one or more of sodium carboxymethyl cellulose, hypromellose, sodium alginate, sodium polyacrylate, sodium hyaluronate, polyethylene glycol, and gelatin. The gel thickener is selected from polyvinyl alcohol. The protectant is selected from a combination of histidine and low-molecular-weight heparin sodium. A combination of the gel matrix and the thickener (polyvinyl alcohol) is used, and on this basis, histidine and low-molecular-weight heparin sodium are used as a protein protectant, so that these auxiliary materials synergistically improve the stability of the recombinant human aFGF gel. It is found through studies that the combination of histidine and heparin sodium can serve as an alternative protein protectant to human serum albumin and can still achieve a good protection effect at a low concentration, thereby effectively reducing the production costs.

### TECHNICAL FIELD

The present invention belongs to the field of pharmaceutical preparations, and particularly relates to a recombinant human acidic fibroblast growth factor gel and a preparation process therefor.

### BACKGROUND ART

An acidic fibroblast growth factor (aFGF) is a member of the fibroblast growth factor (FGF) family that is derived from mesoderm and neuroectoderm. It is a multifunctional cell growth factor and a trace active substance with a wide range of mitogenic effects. It is mainly distributed in organs or tissues such as the brain, pituitary gland, nervous tissue, retina, adrenal gland, heart, and bone, with very low concentrations in other tissues and extremely low concentrations in serum and body fluids. It is an endogenous active factor that is closely related to the healing process of tissue trauma, and has a significant promoting effect on tissue trauma healing, nerve damage repair and vascular regeneration. Such factor is used for the following wounds: deep II degree burn wounds, chronic ulcer wounds (including residual wounds after trauma, diabetic ulcers, vascular ulcers and bedsores), to promote wound healing.

Common dosage forms of acidic fibroblast growth factor (aFGF) include lyophilized powder, eye drops and gels. For lyophilized powder, the solvent provided in the package needs to be poured into the bottle containing rhaFGF lyophilized powder, and after the sprayer head provided in the package is capped (caught), it can be used. When used, the liquid is directly sprayed on the wound after debridement. Eye drops and gels can be used directly. Gels incorporate the characteristics of polymer materials, and can effectively prolong the retention time of added active factors in specific sites, improve drug absorption and bioavailability, have targeted functions, and can slowly release drugs, which can reduce the number of clinical drug administrations and is convenient to use, offering broad market competitiveness.

Gels have the advantages of being easy to spread, comfortable, non-greasy, easy to wash off, able to absorb tissue exudate, not interfering with the normal physiological function of the skin, having a certain water-retaining effect, and promoting the transdermal absorption of drugs. However, acidic fibroblast growth factor (aFGF) aqueous gels are sensitive to external conditions such as temperature, and are prone to denaturation and inactivation when left at room temperature for a long time. In order to solve the above-mentioned technical problems, Chinese invention patent application 202111189805.1 uses Carbomer 940 as a gel matrix, and human serum albumin, sodium heparin, etc. as protective agents to obtain a recombinant human acidic fibroblast growth factor (aFGF) gel that is insensitive to temperature conditions. Although this patent application solves the problem of poor stability of acidic fibroblast growth factor (aFGF) aqueous gels, the use of a large amount of protective agents will undoubtedly increase the cost of the product.

### SUMMARY OF THE INVENTION

In view of the deficiencies of the prior art, the present invention provides a new recombinant human acidic fibroblast growth factor (aFGF) gel to solve the problems of poor product stability and high preparation cost in the prior art.

Specifically, the present invention provides a recombinant human acidic fibroblast growth factor (aFGF) gel, which comprises a gel matrix, a thickener and a protectant, and the gel matrix is selected from one or more of sodium carboxymethyl cellulose, hypromellose, sodium alginate, sodium polyacrylate, sodium hyaluronate, polyethylene glycol, and gelatin, the gel thickener is selected from polyvinyl alcohol, and the protectant is selected from a combination of histidine and heparin sodium.

Further, the present invention provides a recombinant human acidic fibroblast growth factor (aFGF) gel, wherein the protectant is selected from a combination of histidine and heparin sodium, and the heparin sodium is selected from low-molecular-weight heparin sodium, and preferably, the average molecular weight of the low-molecular-weight heparin sodium is 3000 to 8000.

Further, the present invention provides a recombinant human acidic fibroblast growth factor (aFGF) gel, wherein the mass of the histidine is 0.8 to 2‰ of the mass of the gel, preferably 1 to 1.6‰; the mass of the heparin sodium is 0.05 to 1% of the mass of the gel, preferably 0.08 to 0.2‰.

Further, the present invention provides a recombinant human acidic fibroblast growth factor (aFGF) gel, wherein the mass of the gel matrix is 0.5 to 5% of the mass of the gel, preferably 1 to 3%.

Further, the present invention provides a recombinant human acidic fibroblast growth factor (aFGF) gel, wherein the mass of the thickener is 0.5 to 3% of the mass of the gel, preferably 1 to 2%.

Further, the present invention provides a recombinant human acidic fibroblast growth factor (aFGF) gel, which also contains a buffer, and the buffer salt is selected from the group consisting of phosphate buffer system, citrate buffer system, and Tris buffer system, preferably phosphate buffer system, and the preferred pH value of the buffer salt is 6.0 to 7.0, the concentration is 0.01 to 0.05 mol/L, and the preferred concentration is 0.02 mol/L.

Further, the present invention provides a recombinant human acidic fibroblast growth factor (aFGF) gel, wherein the relative amount of the recombinant human acidic fibroblast growth factor in the gel is 2×10³ to 8×10³ IU/g.

Further, the present invention provides a recombinant human acidic fibroblast growth factor (aFGF) gel, which also contains a humectant and a preservative, preferably the humectant is selected from glycerols; the preservative is selected from p-hydroxybenzoate preservatives, preferably the preservative is selected from one or more of methyl p-hydroxybenzoate, ethyl p-hydroxybenzoate, propyl p-hydroxybenzoate, and isopropyl p-hydroxybenzoate; the humectant also has a solubility aid effect.

Further, the present invention provides a recombinant human acidic fibroblast growth factor (aFGF) gel, wherein the mass of the preservative is 0.1 to 3‰ of the mass of the gel, preferably 0.2 to 10‰; and the mass of the humectant is 0.2 to 4% of the mass of the gel, preferably 0.5 to 2%.

The present invention has the following beneficial effects:

The present invention selects a combination of a gel matrix and a thickener (polyvinyl alcohol); on this basis, histidine and heparin sodium are selected as protein protectants. The above auxiliary materials synergistically improve the stability of the recombinant human acidic fibroblast growth factor (aFGF) gel, and the prepared gel product still maintains high stability and relative activity under high temperature conditions.

After screening, it was determined that the combination of histidine and heparin sodium can be used as an alternative protein protectant for human albumin. It can still achieve a high protection effect under lower concentration conditions, effectively reducing production costs.

### DETAILED DESCRIPTION OF EXAMPLES

The present invention is described in detail and specifically by specific examples below to provide a better understanding of the present invention, but the following examples do not limit the scope of the present invention.

The phosphate buffer described in the present invention is Na₂HPO₄, NaH₂PO₄·H₂O buffer, with a concentration of 0.02 mol/L and a pH value of 6.0 to 7.0; the weighing of reagents and the preparation of the formulation are performed under sterile conditions; the average molecular weight of the low-molecular-weight heparin sodium in the examples of the present invention is 3000 to 8000.

Example 1: Taking the preparation of 100 g of recombinant human acidic fibroblast growth factor (aFGF) gel as an example, the specific prescription and preparation method are as follows:

| rhaFGF | 4 × 10⁵ IU | |
|---|---|---|
| Sodium carboxymethyl cellulose | 2 | g |
| Polyvinyl alcohol | 1 | g |
| Histidine | 0.1 | g |
| Low-molecular-weight heparin sodium | 0.02 | g |
| Ethyl p-hydroxybenzoate | 0.05 | g |
| Glycerol | 2 | g |
| NaH₂PO₄•H₂O | 0.189 | g |
| Na₂HPO₄ | 0.089 | g |

(1) Take about 40 ml of water for injection, add the prescribed amount of preservatives and humectants, heat to dissolve and then add the prescribed amount of gel matrix. After fully swelling overnight, sterilize with high-pressure wet heat at 121° C. for 30 minutes, let stand and cool to room temperature, and obtain solution A for use.

(2) Add the prescribed amount of Na₂HPO₄, NaH₂PO₄·H₂O, thickener and protectant into 50 ml of water for injection, respectively, stir to dissolve and then add the prescribed amount of recombinant human acidic fibroblast growth factor stock solution, mix thoroughly and obtain solution B for use.

(3) Filter solution B through a 0.22 μm filter membrane to sterilize it and then add it to solution A. Add the remaining amount of water for injection to make up to 100 g. Stir thoroughly and let stand to obtain rhaFGF gel.

Example 2: Taking the preparation of 100 g of recombinant human acidic fibroblast growth factor (aFGF) gel as an example, the specific prescription is as follows:

| rhaFGF | 6 × 10⁵ IU | |
|---|---|---|
| Hypromellose | 2.5 | g |
| Polyvinyl alcohol | 1 | g |
| Histidine | 0.1 | g |
| Low-molecular-weight heparin sodium | 0.02 | g |
| Methyl p-hydroxybenzoate | 0.05 | g |
| Glycerol | 2 | g |
| NaH₂PO₄•H₂O | 0.189 | g |
| Na₂HPO₄ | 0.089 | g |

The preparation method is as in Example 1.

Example 3: Taking the preparation of 100 g of recombinant human acidic fibroblast growth factor (aFGF) gel as an example, the specific prescription is as follows:

| rhaFGF | 4 × 10⁵ IU | |
|---|---|---|
| Sodium alginate | 4 | g |
| Polyvinyl alcohol | 1.5 | g |
| Histidine | 0.1 | g |
| Low-molecular-weight heparin sodium | 0.02 | g |
| Propyl p-hydroxybenzoate | 0.05 | g |
| Glycerol | 2 | g |
| NaH₂PO₄•H₂O | 0.189 | g |
| Na₂HPO₄ | 0.089 | g |

Comparative Example 1: Taking the preparation of 100 g of recombinant human acidic fibroblast growth factor (aFGF) gel as an example, the specific prescription is as follows:

| rhaFGF | 4 × 10⁵ IU | |
|---|---|---|
| Sodium carboxymethyl cellulose | 2 | g |
| Histidine | 0.1 | g |
| Low-molecular-weight heparin sodium | 0.02 | g |
| Ethyl p-hydroxybenzoate | 0.05 | g |
| Glycerol | 2 | g |
| NaH₂PO₄•H₂O | 0.189 | g |
| Na₂HPO₄ | 0.089 | g |

The preparation method is as in Example 1.

Comparative Example 2: Taking the preparation of 100 g of recombinant human acidic fibroblast growth factor (aFGF) gel as an example, the specific prescription is as follows:

| rhaFGF | 4 × 10⁵ IU | |
|---|---|---|
| Sodium carboxymethyl cellulose | 2 | g |
| Polyvinyl alcohol | 1 | g |
| Low-molecular-weight heparin sodium | 0.02 | g |
| Ethyl p-hydroxybenzoate | 0.05 | g |
| Glycerol | 2 | g |
| NaH₂PO₄•H₂O | 0.189 | g |
| Na₂HPO₄ | 0.089 | g |

The preparation method is as in Example 1.

Comparative Example 3: Taking the preparation of 100 g of recombinant human acidic fibroblast growth factor (aFGF) gel as an example, the specific prescription is as follows:

| rhaFGF | 4 × 10⁵ IU | |
|---|---|---|
| Sodium carboxymethyl cellulose | 2 | g |
| Polyvinyl alcohol | 1 | g |
| Histidine | 0.1 | g |
| Ethyl p-hydroxybenzoate | 0.02 | g |
| Glycerol | 2 | g |
| NaH₂PO₄•H₂O | 0.189 | g |
| Na₂HPO₄ | 0.089 | g |

The prepared rhAFGF gel was subjected to the following experimental verification, and the test results were shown in Table 1.

(1) rhAFGF Gel Stability Test

Take 5 g of the gels of Examples 1-3 and Comparative Examples 1-3, seal them in aluminum tubes, and store them at 25° C. and 40° C., respectively. Check the appearance of the gel regularly, take samples to determine the activity of rhaFGF in the gel, and calculate the relative activity value.

(2) rhAFGF Activity Assay (Using MTT Method)

After re-dissolving the rhAFGF activity assay working standard, use the test culture medium to make three 16-fold dilutions and then seven 4-fold dilutions. Load the last 8 dilution gradients for detection in duplicate wells. After re-dissolving the test sample according to the indicated amount, use the test culture medium to make three 16-fold dilutions and then seven 4-fold dilutions. Load the last 8 dilution gradients for detection in duplicate wells. Complete culture medium was used for incubation and passage of 3T3 cells. 3T3 cells in the logarithmic growth phase were fully digested with trypsin into individual cells and collected, the cell density was adjusted to about 1.0×10⁵ cells/ml with test culture medium to prepare a cell suspension. The cell suspension was inoculated into a 96-well plate with samples in 50 ul per well, and incubated in 37° C. incubator containing 5% CO₂ for 66 to 72 hours. 10 ul MTT solution was added to each well, which was incubated in 37° C. incubator containing 5% CO₂ for 4 to 6 hours. 100 ul 10% SDS was added to each well, which was incubated in 37° C. incubator containing 5% CO₂ overnight. Colorimetry was performed on an ELISA reader at a wavelength of 570 nm or 570 nm/630 nm. The OD value was read and recorded, and the corresponding OD-rhAFGF concentration curve was made. Its activity was calculated according to the following formula:

$$Sample biological activity ( U / ml ) = Pr \times Ds \times Es / ( Dr \times Er )$$

Pr is the biological activity of the standard, in U/ml; and Ds is the pre-dilution factor of the test sample.

Dr is the pre-dilution factor of the standard; and Es is the dilution factor of the test sample equivalent to the half-effective dose of the standard.

Er is the dilution factor of the half-effective dose of the standard.

TABLE 1
Relative activity results of stability study
at 25° C. (relative activity at day 0 is 100%)

| Example/Comparative | Time | | pH | relative |
|---|---|---|---|---|
| Example | (day) | Appearance | value | activity |
| Example 1 | 10 | meet the requirement | 6.5 | 104.1% |
| | 20 | meet the requirement | 6.6 | 99.3% |
| | 30 | meet the requirement | 6.4 | 97.1% |
| | 60 | meet the requirement | 6.5 | 99.4% |
| | 90 | meet the requirement | 6.6 | 100.9% |
| Example 2 | 10 | meet the requirement | 6.5 | 101.1% |
| | 20 | meet the requirement | 6.6 | 103.7% |
| | 30 | meet the requirement | 6.4 | 99.3% |
| | 60 | meet the requirement | 6.5 | 98.6% |
| | 90 | meet the requirement | 6.6 | 102.1% |
| Example 3 | 10 | meet the requirement | 6.6 | 99.4% |
| | 20 | meet the requirement | 6.5 | 104.8% |
| | 30 | meet the requirement | 6.4 | 101.2% |
| | 60 | meet the requirement | 6.5 | 97.8% |
| | 90 | meet the requirement | 6.4 | 99.2% |
| Comparative Example 1 | 10 | meet the requirement | 6.5 | 97.1% |
| | 20 | meet the requirement | 6.5 | 84.9% |
| | 30 | meet the requirement | 6.6 | 76.9% |
| | 60 | meet the requirement | 6.5 | 69.4% |
| | 90 | meet the requirement | 6.4 | 57.6% |
| Comparative Example 2 | 10 | meet the requirement | 6.6 | 100.8% |
| | 20 | meet the requirement | 6.5 | 96.7% |
| | 30 | meet the requirement | 6.7 | 88.4% |
| | 60 | meet the requirement | 6.5 | 80.2% |
| | 90 | meet the requirement | 6.4 | 76.2% |
| Comparative Example 3 | 10 | meet the requirement | 6.5 | 92.1% |
| | 20 | meet the requirement | 6.6 | 81.7% |
| | 30 | meet the requirement | 6.5 | 73.2% |
| | 60 | meet the requirement | 6.4 | 57.5% |
| | 90 | meet the requirement | 6.5 | 38.6% |

TABLE 2
Relative activity results of stability study
at 40° C. (relative activity at day 0 is 100%)

| Example/Comparative | Time | | pH | relative |
|---|---|---|---|---|
| Example | (day) | Appearance | value | activity |
| Example 1 | 10 | meet the requirement | 6.6 | 103.1% |
| | 20 | meet the requirement | 6.5 | 97.3% |
| | 30 | meet the requirement | 6.7 | 98.1% |
| | 60 | meet the requirement | 6.5 | 99.4% |
| | 90 | meet the requirement | 6.4 | 97.9% |
| Example 2 | 10 | meet the requirement | 6.5 | 99.4% |
| | 20 | meet the requirement | 6.5 | 103.2% |
| | 30 | meet the requirement | 6.4 | 100.8% |
| | 60 | meet the requirement | 6.5 | 96.8% |
| | 90 | meet the requirement | 6.6 | 99.2% |
| Example 3 | 10 | meet the requirement | 6.5 | 97.4% |
| | 20 | meet the requirement | 6.5 | 99.8% |
| | 30 | meet the requirement | 6.4 | 100.2% |
| | 60 | meet the requirement | 6.5 | 96.9% |
| | 90 | meet the requirement | 6.6 | 98.4% |
| Comparative Example 1 | 10 | meet the requirement | 6.6 | 104.1% |
| | 20 | meet the requirement | 6.5 | 88.1% |
| | 30 | meet the requirement | 6.4 | 77.7% |
| | 60 | meet the requirement | 6.5 | 70.1% |
| | 90 | meet the requirement | 6.4 | 58.4% |
| Comparative Example 2 | 10 | meet the requirement | 6.5 | 96.6% |
| | 20 | meet the requirement | 6.4 | 82.4% |
| | 30 | meet the requirement | 6.5 | 72.1% |
| | 60 | meet the requirement | 6.6 | 58.8% |
| | 90 | meet the requirement | 6.5 | 49.6% |
| Comparative Example 3 | 10 | meet the requirement | 6.7 | 82.3% |
| | 20 | meet the requirement | 6.6 | 67.3% |
| | 30 | meet the requirement | 6.5 | 43.5% |
| | 60 | meet the requirement | 6.5 | 26.2% |
| | 90 | meet the requirement | 6.5 | 14.6% |

The above contents cannot be used to determine that the specific implementation of the present invention is limited to these descriptions. Those skilled in the art to which the present invention belongs can make several simple deductions or substitutions without departing from the concept of the present invention, which should be regarded as belonging to the protection scope of the patent of the present invention determined by the submitted claims.

1. A recombinant human acidic fibroblast growth factor (aFGF) gel, wherein the gel comprises a gel matrix, a thickener, and a protectant, and the gel matrix is selected from one or more of sodium carboxymethyl cellulose, hypromellose, sodium alginate, sodium polyacrylate, sodium hyaluronate, polyethylene glycol, and gelatin, the gel thickener is selected from polyvinyl alcohol, and the protectant is selected from a combination of histidine and low-molecular-weight heparin sodium; wherein the low-molecular-weight heparin sodium has an average molecular weight of 3000 to 8000, wherein the mass of the gel matrix comprises 0.5 to 5.0% of the mass of the gel, wherein the mass of the thickener comprises 0.5 to 3.0% of the mass of the gel, wherein the mass of the histidine comprises 0.8 to 2‰ of the mass of the gel, and wherein the mass of the low-molecular-weight heparin sodium comprises 0.05 to 1‰ of the mass of the gel.

2. The recombinant human acidic fibroblast growth factor (aFGF) gel according to claim 1, wherein the mass of the gel matrix comprises 1.0 to 3.0% of the mass of the gel.

3. The recombinant human acidic fibroblast growth factor (aFGF) gel according to claim 1, wherein the mass of the thickener comprises 1 to 2% of the mass of the gel.

4. The recombinant human acidic fibroblast growth factor (aFGF) gel according to claim 1, wherein the mass of the histidine comprises 1 to 1.6‰ of the mass of the gel.

5. The recombinant human acidic fibroblast growth factor (aFGF) gel according to claim 1, wherein the gel further comprises a buffer, and the buffer is selected from the group consisting of phosphate buffer system, citrate buffer system, and Tris buffer system, and wherein the buffer has a pH value of 6.0 to 7.0, the buffer has a concentration of 0.01 to 0.05 mol/L, or the buffer has a pH value of 6.0 to 7.0 and a concentration of 0.01 to 0.05 mol/L.

6. The recombinant human acidic fibroblast growth factor (aFGF) gel according to claim 1, wherein the relative amount of recombinant human acidic fibroblast growth factor in the gel is 2×10³ to 8×10³ IU/g.

7. The recombinant human acidic fibroblast growth factor (aFGF) gel according to claim 1, wherein the gel further contains a humectant and a preservative.

8. The recombinant human acidic fibroblast growth factor (aFGF) gel according to claim 7, wherein the humectant is selected from glycerols; and the preservative is selected from p-hydroxybenzoate preservatives.

9. The recombinant human acidic fibroblast growth factor (aFGF) gel according to claim 7, wherein the preservative is selected from one or more of methyl p-hydroxybenzoate, ethyl p-hydroxybenzoate, propyl p-hydroxybenzoate, and isopropyl p-hydroxybenzoate.

10. The recombinant human acidic fibroblast growth factor (aFGF) gel according to claim 7, wherein the mass of the preservative comprises 0.1 to 3‰ of the mass of the gel; and the mass of the humectant comprises 0.2 to 4% of the mass of the gel.

11. The recombinant human acidic fibroblast growth factor (aFGF) gel according to claim 1, wherein the mass of the low-molecular-weight heparin sodium comprises 0.08 to 0.2%, of the mass of the gel.

12. The recombinant human acidic fibroblast growth factor (aFGF) gel according to claim 5, wherein the buffer is selected from phosphate buffer system, the buffer has a pH value of 6.0 to 7.0 and a concentration is 0.02 mol/L, or the buffer is selected from phosphate buffer system and has a pH value of 6.0 to 7.0 and a concentration is 0.02 mol/L.

13. The recombinant human acidic fibroblast growth factor (aFGF) gel according to claim 7, wherein the mass of the preservative comprises 0.2 to 1‰ of the mass of the gel.

14. The recombinant human acidic fibroblast growth factor (aFGF) gel according to claim 7, wherein the mass of the humectant comprises 0.5 to 2% of the mass of the gel.
